(19) United States
(12) Patent Application Publication
(10) Pub. No.: US 2025/0154103 A1
(43) Pub. Date: May 15, 2025

(54) A process for the preparation of Triclopyr-Butotyl

(71) Applicant: GHARDA CHEMICALS LIMITED, Ratnagiri, Maharashtra (IN)

(72) Inventors: Diwakar K. SHENOY, Ratnagiri, Maharashtra (IN); Laxminarayan S. SHET, Ratnagiri, Maharashtra (IN); Yatin S. SAMANGADKAR, Ratnagiri, Maharashtra (IN); Sandesh S. THASALE, Ratnagiri, Maharashtra (IN); Manoj V. SALVI, Ratnagiri, Maharashtra (IN); Sharad Y. MANE, Ratnagiri, Maharashtra (IN); Sandeep S. KEDARE, Ratnagiri, Maharashtra (IN); Nilesh R. SAWANT, Ratnagiri, Maharashtra (IN); Vinay V. KALASAPUR, Ratnagiri, Maharashtra (IN); Sachin B. JADHAV, Ratnagiri, Maharashtra (IN)

(21) Appl. No.: 18/839,196

(22) PCT Filed: Feb. 15, 2023
(86) PCT No.: PCT/IB2023/051350

§ 371 Date: Aug. 16, 2024

(30) Foreign Application Priority Data
Feb. 18, 2022 (IN) 202221008648
Feb. 18, 2022 (IN) 202221008650
Feb. 18, 2022 (IN) 202221008651

(51) Int. Cl. C07D 213/64; B01D 3/10; B01D 3/14; B01D 3/36

(52) U.S. Cl. CPC C07D 213/64; B01D 3/10; B01D 3/143; B01D 3/36

(57) ABSTRACT

The present disclosure relates to a process for the preparation of triclopyr-butotyl. Further, the present disclosure relates to a process for the preparation of 2-butoxyethyl chloroacetate. The fluid medium and the acid solution used in the process of the present disclosure can be recovered and recycled making the process environment friendly as well as economical. The process of the present disclosure provides triclopyr-butotyl with a comparatively high purity and high yield.

### FIELD

The present disclosure relates to a process for the preparation of triclopyr-butotyl.

### BACKGROUND

The background information herein below relates to the present disclosure but is not necessarily prior art.

Triclopyr-butotyl is an organic compound in the pyridine group that is used as a systemic foliar herbicide and fungicide. Triclopyr-butotyl is used to control broadleaf weeds and to control rust diseases on crops. The chemical name of triclopyr-butotyl is 2-[(3,5,6-trichloro-2-pyridinyl)oxy]acetic acid 2-butoxyethyl ester and has the following chemical structure:

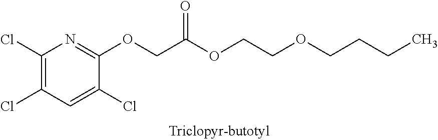 [SMILES: CCCCOCCOC(=O)COc1nc(Cl)c(Cl)cc1Cl]

Conventionally, the process of preparing triclopyr-butotyl involves oxidation of pyridine and quinoline bases. However, the conventional process consumes more energy and results in greater loss of solvent during the reaction. Moreover, the conventional processes results in lower yield and lower purity of triclopyr-butotyl.

Further, 2-butoxyethyl chloroacetate is an important intermediate used for the preparation of Triclopyr-butotyl-butotyl. Conventionally, the process of preparing 2-butoxyethyl chloroacetate involves catalytic esterification of butyl cellosolve and mono chloro acetic acid by using mineral acid as a catalyst. However, various impurities/by-products are formed during the conventional processes. Further, the use of mineral acids on a large scale production/reaction is not convenient due to the corrosive nature of the mineral acids.

Therefore, there is, felt a need to provide a process for preparing triclopyr-butotyl and 2-butoxyethyl chloroacetate that mitigates the aforestated drawbacks or at least provides a useful alternative.

### Objects

Some of the objects of the present disclosure, which at least one embodiment herein satisfies, are as follows:

It is an object of the present disclosure to ameliorate one or more problems of the background or to at least provide a useful alternative.

An object of the present disclosure is to provide a process for the preparation of triclopyr-butotyl.

Another object of the present disclosure is to provide a process for the preparation of triclopyr-butotyl with high yield and high purity.

Yet another object of the present disclosure is to provide a process for the preparation of triclopyr-butotyl that is environment friendly.

Still another object of the present disclosure is to provide a simple and cost-efficient process for the preparation of triclopyr-butotyl.

Another object of the present disclosure is to provide a simple, cost-efficient and environment friendly process for the preparation of 2-butoxyethyl chloroacetate.

Other objects and advantages of the present disclosure will be more apparent from the following description, which is not intended to limit the scope of the present disclosure.

### SUMMARY

The present disclosure relates to a process for the preparation of triclopyr-butotyl. The process comprises reacting 2-butoxyethyl chloroacetate with a compound selected from sodium 3,5,6-trichloropyridin-2-olate, potassium 3,5,6-trichloropyridin-2-olate and ammonium 3,5,6-trichloropyridin-2-olate in a predetermined molar ratio by using a first base in the presence of a first catalyst under stirring at a first predetermined temperature for a first predetermined time period to obtain a reaction mixture. A predetermined amount of first fluid medium is added to the reaction mixture at a second predetermined temperature followed by cooling to a third predetermined temperature to obtain a reaction mass. The reaction mass is filtered to obtain a residue comprising insoluble impurities and a salt; and a filtrate comprising triclopyr-butotyl. The filtrate is cooled to a fourth predetermined temperature to obtain a cooled filtrate. The cooled filtrate is washed with a predetermined concentration of sulfuric acid for a second predetermined time period to obtain a biphasic mixture containing a top organic layer comprising triclopyr-butotyl and a bottom aqueous layer comprising sulfuric acid solution. The aqueous layer comprising the acid solution is separated to obtain the organic layer comprising the triclopyr-butotyl and the first fluid medium. The organic layer is washed with at least one salt solution and a second base solution to obtain a washed organic layer comprising the triclopyr-butotyl and the first fluid medium. The washed organic layer is distilled at a fifth predetermined temperature to recover the first fluid medium, to obtain a residual mass containing the triclopyr-butotyl. The residual mass is vacuum dried at a sixth predetermined temperature and at a predetermined pressure to obtain the triclopyr-butotyl.

The present disclosure further relates to a process for the preparation of 2-butoxyethyl chloroacetate. The process comprises reacting butyl cellosolve with mono chloro acetic acid in a predetermined molar ratio in the presence of a second catalyst in a second fluid medium to obtain a reaction mixture. The reaction mixture is heated at a seventh predetermined temperature and simultaneously water is azeotropically distilled out to obtain a first product mixture comprising 2-butoxyethyl chloroacetate, the second fluid medium, unreacted butyl cellosolve, unreacted mono chloro acetic acid and the second catalyst. 2-butoxyethyl chloroacetate is isolated and purified from said first product mixture.

### DETAILED DESCRIPTION

The present disclosure relates to a process for the preparation of triclopyr-butotyl.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise.

The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Triclopyr-butotyl is an organic compound in the pyridine group that is used as a systemic foliar herbicide and fungicide. Triclopyr-butotyl is used to control broadleaf weeds and to control rust diseases on crops.

Conventionally, the process of preparing triclopyr-butotyl involves oxidation of pyridine and quinoline bases. However, the conventional process consumes more energy and results in greater loss of solvent during the reaction. Moreover, the conventional processes results in lower yield and lower purity of triclopyr-butotyl.

Further, 2-butoxyethyl chloroacetate is an important intermediate used for the preparation of Triclopyr-butotyl-butotyl. Conventionally, the process of preparing 2-butoxyethyl chloroacetate involves catalytic esterification of butyl cellosolve and mono chloro acetic acid by using mineral acid as a catalyst. However, various impurities/by-products are formed during the conventional processes. Further, the use of mineral acids on a large scale production/reaction is not convenient due to the corrosive nature of the mineral acids.

The present disclosure provides a simple, cost-efficient and environmental friendly processes for the preparation of triclopyr-butotyl and 2-butoxyethyl chloroacetate.

In an aspect, the present disclosure provides a process for the preparation of triclopyr-butotyl. Triclopyr-butotyl is represented below as Formula I.

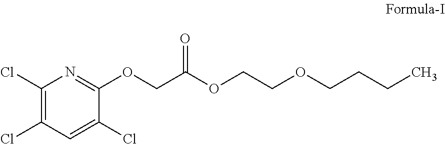 [SMILES: CCCCOCCOC(=O)COc1nc(Cl)c(Cl)cc1Cl]

Chemical formula: C₁₃H₁₆Cl₃NO₄

Molar mass: 356.6

The process for the preparation of triclopyr-butotyl comprises the following steps:
i. reacting 2-butoxyethyl chloroacetate with a compound selected from sodium 3,5,6-trichloropyridin-2-olate, potassium 3,5,6-trichloropyridin-2-olate and ammonium 3,5,6-trichloropyridin-2-olate in a predetermined molar ratio by using a first base in the presence of a first catalyst under stirring at a first predetermined temperature for a first predetermined time period to obtain a reaction mixture;
ii. adding a predetermined amount of first fluid medium to the reaction mixture at a second predetermined temperature followed by cooling to a third predetermined temperature to obtain a reaction mass;
iii. filtering the reaction mass to obtain a residue comprising insoluble impurities and a salt; and a filtrate comprising triclopyr-butotyl;
iv. cooling the filtrate to a fourth predetermined temperature to obtain a cooled filtrate;
v. washing the cooled filtrate with a predetermined concentration of sulfuric acid for a second predetermined time period to obtain a biphasic mixture containing a top organic layer comprising triclopyr-butotyl and a bottom aqueous layer comprising sulfuric acid solution;
vi. separating the aqueous layer comprising the acid solution to obtain the organic layer comprising the triclopyr-butotyl and the first fluid medium;
vii. washing the organic layer with at least one salt solution and a second base solution to obtain a washed organic layer comprising the triclopyr-butotyl and the first fluid medium;
viii. distilling the washed organic layer at a fifth predetermined temperature to recover the first fluid medium, to obtain a residual mass containing the triclopyr-butotyl; and
ix. vacuum drying the residual mass at a sixth predetermined temperature and at a predetermined pressure to obtain the triclopyr-butotyl.

The process is described in detail herein below:

In a first step, 2-butoxyethyl chloroacetate is reacted with a compound selected from sodium 3,5,6-trichloropyridin-2-olate, potassium 3,5,6-trichloropyridin-2-olate and ammonium 3,5,6-trichloropyridin-2-olate in a predetermined molar ratio by using a first base in the presence of a first catalyst under stirring at a first predetermined temperature for a first predetermined time period to obtain a reaction mixture.

In an exemplary embodiment of the present disclosure, the compound is sodium 3,5,6-trichloropyridin-2-olate.

In an embodiment of the present disclosure, the predetermined molar ratio of 2-butoxyethyl chloroacetate to sodium 3,5,6-trichloropyridin-2-olate is in the range of 1:1 to 1:1.5. In an exemplary embodiment of the present disclosure, the molar ratio of 2-butoxyethyl chloroacetate to sodium 3,5,6-trichloropyridin-2-olate is 1:1.05.

The first catalyst is selected from tetrabutylammonium bromide (TBAB) and benzyl triethyl ammonium chloride. In an exemplary embodiment of the present disclosure, the first catalyst is tetrabutylammonium bromide (TBAB). In another exemplary embodiment of the present disclosure, the first catalyst is benzyl triethyl ammonium chloride.

The first base is selected from sodium carbonate, sodium bicarbonate, potassium carbonate, potassium bicarbonate, sodium hydroxide and potassium hydroxide. In an exemplary embodiment of the present disclosure, the first base is sodium bicarbonate.

The first predetermined temperature is in the range of 45° C. to 90° C. In an exemplary embodiment of the present disclosure, the first predetermined temperature is 77° C. In another exemplary embodiment of the present disclosure, the first predetermined temperature is 75° C. In still another exemplary embodiment of the present disclosure, the first predetermined temperature is 65° C. In yet another exemplary embodiment of the present disclosure, the first predetermined temperature is 50° C.

The first predetermined time period is in the range of 3 hours to 20 hours. In an exemplary embodiment of the present disclosure, the first predetermined time period is 4 hours. In another exemplary embodiment of the present disclosure, the first predetermined time period is 6 hours. In still another exemplary embodiment of the present disclosure, the first predetermined time period is 10 hours. In yet another exemplary embodiment of the present disclosure, the first predetermined time period is 16 hours.

In a second step, a predetermined amount of a first fluid medium is added to the reaction mixture at a second predetermined temperature followed by cooling to a third predetermined temperature to obtain a reaction mass.

The second predetermined temperature is in the range of 45° C. to 60° C. In an exemplary embodiment of the present disclosure, the second predetermined temperature is 60° C. In another exemplary embodiment of the present disclosure, the second predetermined temperature is 50° C.

The first fluid medium is selected from the group consisting of toluene, n-hexane and benzene. In an exemplary embodiment of the present disclosure, the first fluid medium is n-hexane.

The predetermined amount of the first fluid medium is in the range of 300 ml/mole of 2-butoxyethyl chloroacetate to 1000 ml/mole of 2-butoxyethyl chloroacetate. In an exemplary embodiment of the present disclosure, the predetermined amount of the first fluid medium is 400 ml/mole of 2-butoxyethyl chloroacetate.

The third predetermined temperature is in the range of 25° C. to 35° C. In an exemplary embodiment of the present disclosure, the third predetermined temperature is 30° C.

In a third step, the reaction mass is filtered to obtain a residue comprising insoluble impurities and a salt; and a filtrate comprising triclopyr-butotyl.

In an embodiment of the present disclosure, the salt is sodium chloride.

In a fourth step, the filtrate is cooled to a fourth predetermined temperature to obtain a cooled filtrate.

The fourth predetermined temperature is in the range of 0° C. to 20° C. In an exemplary embodiment of the present disclosure, the fourth predetermined temperature is 15° C.

In a fifth step, the cooled filtrate is washed with a predetermined concentration of sulfuric acid for a second predetermined time period to obtain a biphasic mixture containing a top organic layer comprising triclopyr-butotyl and a bottom aqueous layer comprising sulfuric acid solution.

The predetermined concentration of sulfuric acid is in the range of 60% w/w to 80% w/w. In an exemplary embodiment of the present disclosure, the predetermined concentration of sulfuric acid is 70% w/w.

The second predetermined time period is in the range of 20 minutes to 60 minutes. In an exemplary embodiment of the present disclosure, the second predetermined time period is 30 minutes.

In a sixth step, the aqueous layer comprising the acid solution is separated to obtain the organic layer comprising the triclopyr-butotyl and the first fluid medium.

In accordance with an embodiment of the present disclosure, the spent sulfuric acid generated in the process is diluted to 50% w/w and extracted with toluene to remove organic impurities in it. The diluted and cleaned H₂SO₄ is concentrated to get 80% w/w and recycled in the process, Thus, the process of the present disclosure is economical and environment friendly.

In a seventh step, the organic layer is washed with at least one salt solution and a second base solution to obtain a washed organic layer comprising the triclopyr-butotyl and the first fluid medium.

The salt solution is prepared by using an alkali metal salt selected from sodium hypochlorite (NaOCl), sodium bisulfite (NaHSO₃) and sodium sulfite (Na₂SO₃). In an exemplary embodiment of the present disclosure, the alkali metal salt is sodium hypochlorite. In another exemplary embodiment of the present disclosure, the alkali metal salt is sodium bisulfite.

The second base is selected from sodium carbonate, sodium bicarbonate, potassium carbonate, potassium bicarbonate, sodium hydroxide and potassium hydroxide. In an exemplary embodiment of the present disclosure, the second base is sodium bicarbonate.

In an embodiment of the present disclosure, the first base and the second base are same.

In another embodiment of the present disclosure, the first base and the second base are different.

In accordance with an embodiment of the present disclosure, the organic layer is washed with the sodium hypochlorite solution and the sodium bicarbonate solution followed by washing with the sodium bisulfite solution to obtain a washed organic layer.

In an eighth step, the washed organic layer is distilled at a fifth predetermined temperature to recover the first fluid medium, to obtain a residual mass containing the triclopyr-butotyl.

The fifth predetermined temperature is in the range of 60° C. to 120° C. In an exemplary embodiment of the present disclosure, the fifth predetermined temperature is 100° C.

In a ninth step, the residual mass is vacuum dried at a sixth predetermined temperature and at a predetermined pressure to obtain the triclopyr-butotyl.

The sixth predetermined temperature is in the range of 90° C. to 120° C. In an exemplary embodiment of the present disclosure, the sixth predetermined temperature is 100° C.

The predetermined pressure is in the range of 4 mmHg to 8 mmHg. In an embodiment of the present disclosure, the predetermined pressure is in the range of 5 mmHg to 7 mmHg. In an exemplary embodiment of the present disclosure, the predetermined pressure is 6 mmHg.

In accordance with an embodiment of the present disclosure, the recovered first fluid medium is recycled and used in the next batch, thus, making the process economical and environment friendly.

The process of the present disclosure is simple, economical, environment friendly and suitable for industrial applications.

The yield of triclopyr-butotyl obtained by the process of the present disclosure is in the range of 85 mole % to 90 mole %. The purity of 2-butoxyethyl chloroacetate obtained by the process of the present disclosure is 98.0% to 99%.

The catalyst, tetrabutylammonium bromide (TBAB) used in the process of the present disclosure is a novel catalyst for preparing triclopyr-butotyl. The use of this catalyst leads to the production of triclopyr-butotyl in significantly higher yield and high purity. Further, when the catalyst loading is more, the reaction rate becomes faster even at low temperature (50° C. to 65° C.) whereas, when the temperature is raised to 77° C. the catalyst loading required is less.

In another aspect, the present disclosure provides a process for the preparation of 2-Butoxyethyl chloroacetate. 2-Butoxyethyl chloroacetate is represented below as Formula II.

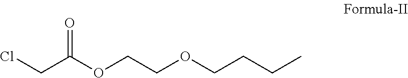

Chemical formula: C₈H₁₅ClO₃

Molar mass: 194.65

The process for the preparation of 2-butoxyethyl chloroacetate comprises the following steps:
i. reacting butyl cellosolve with mono chloro acetic acid in a predetermined molar ratio in the presence of a second catalyst in a second fluid medium to obtain a reaction mixture;
ii. heating the reaction mixture at a seventh predetermined temperature and simultaneously distilling out water azeotropically to obtain a first product mixture comprising 2-butoxyethyl chloroacetate, the second fluid medium, unreacted butyl cellosolve, unreacted mono chloro acetic acid and the second catalyst; and
iii. isolating and purifying 2-butoxyethyl chloroacetate from the first product mixture.

The process is described in detail herein below:

During the process, firstly, butyl cellosolve is reacted with mono chloro acetic acid in a predetermined molar ratio in the presence of a second catalyst in a second fluid medium to obtain a reaction mixture.

In an embodiment of the present disclosure, the predetermined molar ratio of the butyl cellosolve to the mono chloro acetic acid is in the range of 1:0.5 to 1:1.5. In an exemplary embodiment of the present disclosure, the molar ratio of the butyl cellosolve to the mono chloro acetic acid is 1:0.9.

The second catalyst is selected from the group consisting of para toluene sulfonic acid (PTSA) and methane sulphonic acid. In an exemplary embodiment of the present disclosure, the second catalyst is para toluene sulfonic acid (PTSA).

The second catalyst is present in an amount in the range of 1 mole % to 2 mole % with respect to the amount of butyl cellosolve. In an exemplary embodiment of the present disclosure, the amount of the second catalyst is 1 mole % with respect to the amount of butyl cellosolve.

The second fluid medium is selected from the group consisting of toluene, n-hexane, cyclohexane, benzene and chlorobenzene. In an exemplary embodiment of the present disclosure, the second fluid medium is toluene. In another exemplary embodiment of the present disclosure, the second fluid medium is n-hexane.

Further, the reaction mixture is heated at a seventh predetermined temperature and simultaneously water is distilled out azeotropically to obtain a first product mixture comprising 2-butoxyethyl chloroacetate, the second fluid medium, unreacted butyl cellosolve, unreacted mono chloro acetic acid and the second catalyst.

The seventh predetermined temperature is in the range of 50° C. to 130° C. In an exemplary embodiment of the present disclosure, the seventh predetermined temperature is 86° C. In another exemplary embodiment of the present disclosure, the seventh predetermined temperature is 65° C.

Finally the so obtained 2-butoxyethyl chloroacetate is isolated and purified from the first product mixture.

The isolation and the purification of 2-butoxyethyl chloroacetate are carried out by;

either;
a) fractionally distilling the first product mixture at an eighth pre-determined temperature to recover the second fluid medium and the unreacted butyl cellosolve, to obtain a second product mixture comprising 2-butoxyethyl chloroacetate, the unreacted mono chloro acetic acid and the second catalyst; and
b) vacuum distilling the second product mixture at a ninth predetermined temperature and at a first predetermined pressure to obtain 2-butoxyethyl chloroacetate and a residual mass containing the second catalyst and the unreacted mono chloroacetic acid;
   or;
a) adding water to the first product mixture to obtain a first biphasic mixture containing a first organic phase and a first aqueous phase, wherein the first aqueous phase comprises the unreacted mono chloro acetic acid and the second catalyst and the first organic phase comprises 2-butoxyethyl chloroacetate and the second fluid medium;
b) separating the first aqueous phase from the first biphasic mixture to obtain the first organic phase comprising 2-butoxyethyl chloroacetate and the second fluid medium;
c) washing the first organic phase with an aqueous third base by maintaining a pH in the range of 6 to 9 to obtain a second biphasic mixture containing a second organic phase and a second aqueous phase followed by separating the second aqueous phase from the second biphasic mixture to obtain the second organic phase containing 2-butoxyethyl chloroacetate and the second fluid medium; and
d) vacuum distilling the second organic phase at a tenth predetermined temperature and at a second predetermined pressure to recover the second fluid medium, to obtain a residual mass containing 2-butoxyethyl chloroacetate.

### Isolation and Purification of 2-Butoxyethyl Chloroacetate (Green Chemistry)

In an embodiment of the present disclosure, the isolation and the purification of 2-butoxyethyl chloroacetate are carried out by fractionally distilling the first product mixture at an eighth pre-determined temperature to recover the second fluid medium and the unreacted butyl cellosolve, to obtain a second product mixture comprising 2-butoxyethyl chloroacetate, the unreacted mono chloro acetic acid and the second catalyst. The second product mixture is vacuum distilling at a ninth predetermined temperature and at a first predetermined pressure to obtain 2-butoxyethyl chloroacetate and a residual mass containing the second catalyst and the unreacted mono chloroacetic acid;

The eighth pre-determined temperature is in the range of 90° C. to 130° C. In an exemplary embodiment of the present disclosure, the eighth predetermined temperature is 130° C. In another exemplary embodiment of the present disclosure, the eighth predetermined temperature is 125° C. In still another exemplary embodiment of the present disclosure, the eighth predetermined temperature is 95° C.

In accordance with an embodiment of the present disclosure, during the fractional distillation of the first product mixture, traces of 2-butoxy chloroacetate is also distilled out along with the second fluid medium and the unreacted butyl cellosolve.

The second fluid medium, the unreacted butyl cellosolve and 2-butoxy chloroacetate are recycled and used in the next batch thereby making the process economical and environment friendly.

The ninth pre-determined temperature is in the range of 100° C. to 120° C. In an exemplary embodiment of the present disclosure, the ninth predetermined temperature is 110° C.

The first pre-determined pressure is in the range of 4 mmHg to 5 mmHg. In an exemplary embodiment of the present disclosure, the first pre-determined pressure is 4.5 mmHg.

In accordance with an embodiment of the present disclosure, the residual mass containing the second catalyst and the unreacted mono chloroacetic acid is recycled and used in the next batch, thus, making the process economical and environment friendly.

In accordance with the embodiment of the present disclosure, the process for the preparation of 2-butoxyethyl chloroacetate is simple, economical, environment friendly and suitable for industrial applications. The unreacted starting materials, the fluid medium and the catalysts are recovered and recycled in the next batch that makes the process of the present disclosure economical and environment friendly.

In accordance with the embodiment of the present disclosure, the yield of 2-butoxyethyl chloroacetate obtained by the process of the present disclosure is in the range of 90 mole % to 98 mole %. The purity of 2-butoxyethyl chloroacetate obtained by the process of the present disclosure is 98.0% to 99.9%.

### Isolation and Purification of 2-Butoxyethyl Chloroacetate by Aqueous Work-Up:

In another embodiment of the present disclosure, the isolation and the purification of 2-butoxyethyl chloroacetate are carried out by adding water to the first product mixture to obtain a first biphasic mixture containing a first organic phase and a first aqueous phase, wherein the first aqueous phase comprises the unreacted mono chloro acetic acid and the second catalyst and the first organic phase comprises 2-butoxyethyl chloroacetate and the second fluid medium. The first aqueous phase is separated from the first biphasic mixture to obtain the first organic phase comprising 2-butoxyethyl chloroacetate and the second fluid medium. The first organic phase is washed with an aqueous third base by maintaining a pH in the range of 6 to 9 to obtain a second biphasic mixture containing a second organic phase and a second aqueous phase followed by separating the second aqueous phase from the second biphasic mixture to obtain the second organic phase containing 2-butoxyethyl chloroacetate and the second fluid medium. The second organic phase is vacuum distilled at a tenth predetermined temperature and at a second predetermined pressure to recover the second fluid medium, to obtain a residual mass containing 2-butoxyethyl chloroacetate.

The advantage of adding water to the first product mixture is that the majority of the unreacted mono chloro acetic acid and the second catalyst get dissolved in water and get separated from the first product mixture and can be recycled in the next batch. Therefore, a less amount of third base will be required in the next step for washing off the traces of acids present in the first organic phase.

In accordance with an embodiment of the present disclosure, the first aqueous phase containing the second catalyst and the unreacted mono chloro acetic acid are recycled and used in the next batch, thus, making the process economical and environment friendly.

The aqueous third base neutralizes the traces of acids present in the first organic phase to form salts. Thus, the second aqueous phase comprises the traces of mono chloro acetic acid salt and traces of second catalyst salt which is separated from the second biphasic mixture. Thus, the acid impurities are completely removed from the second organic phase containing 2-butoxyethyl chloroacetate and the second fluid medium.

The third base is selected from the group consisting of sodium carbonate, sodium bicarbonate, potassium carbonate, potassium bicarbonate, sodium hydroxide and potassium hydroxide. In an exemplary embodiment of the present disclosure, the third base is sodium carbonate.

In an embodiment of the present disclosure, the pH is in the range of 7 to 8. In an exemplary embodiment of the present disclosure, the pH is 7.5.

The tenth predetermined temperature is in the range of 100° C. to 130° C. In an exemplary embodiment of the present disclosure, the tenth predetermined temperature is 105° C. In another exemplary embodiment of the present disclosure, the tenth predetermined temperature is 110° C.

The second predetermined pressure is in the range of 10 mmHg to 20 mmHg. In an exemplary embodiment of the present disclosure, the second predetermined pressure is 15 mmHg.

In accordance with another embodiment of the present disclosure, during the vacuum distillation of the organic phase, traces of 2-butoxy chloroacetate is also distilled out along with the second fluid medium.

In accordance with the embodiment of the present disclosure, the yield of 2-butoxyethyl chloroacetate obtained by the process of the present disclosure is in the range of 90 mole % to 99 mole %. The purity of 2-butoxyethyl chloroacetate obtained by the process of the present disclosure is 98.0% to 99.9%.

In accordance with the second embodiment of the present disclosure, the recovered second fluid medium along with the traces of 2-butoxy chloroacetate are recycled and used in the next batch making the process economical and environment friendly.

In accordance with the second embodiment of the present disclosure, the process for the preparation of 2-butoxyethyl chloroacetate is simple, economical, environment friendly and suitable for industrial applications. The unreacted starting materials, fluid medium and catalysts are recovered and recycled in the next batch making the process of the present disclosure economical and environment friendly.

The foregoing description of the embodiments has been provided for purposes of illustration and is not intended to limit the scope of the present disclosure. Individual components of a particular embodiment are generally not limited to that particular embodiment but are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments can be scaled up to industrial/commercial scale and the results obtained can be extrapolated to the industrial scale.

### Experimental Details

### Experiment I: Preparation of 2-Butoxyethyl Chloroacetate (Green Chemistry)

### Example 1: Preparation of 2-Butoxyethyl Chloroacetate by Using Toluene as a Fluid Medium in Accordance with the Present Disclosure

In 4 neck round bottom flask with Dean and Stark apparatus, double coil condenser, overhead stirrer and reaction temperature indicator, 129.8 g of butyl cellosolve and 94.5 g of mono chloro acetic acid were charged and reacted in the presence of 2.5 g of para toluene sulfonic acid (PTSA) in 100 ml toluene to obtain a reaction mixture. The reaction mixture was heated at 86° C. and simultaneously water was removed through the Dean and Stark system to obtain a first product mixture comprising 2-butoxyethyl chloroacetate, toluene, unreacted butyl cellosolve, unreacted mono chloro acetic acid and para toluene sulfonic acid. After complete removal of water (18 ml), the first product mixture was fractionally distilled at 130° C. to recover toluene, and unreacted butyl cellosolve, to obtain a second product mixture comprising 2-butoxyethyl chloroacetate, unreacted mono chloro acetic acid and the para toluene sulfonic acid. Small fraction of 2-butoxyethyl chloroacetate was also distilled out along with toluene and unreacted butyl cellosolve. The second product mixture was vacuum distilled at 110° C. and at a pressure of 4.5 mmHg to obtain 2-butoxyethyl chloroacetate and a residual mass containing the para toluene sulfonic acid and the unreacted mono chloroacetic acid. Residual mass containing the para toluene sulfonic acid and the unreacted mono chloroacetic acid was recycled and used in the next batch.

The purity of 2-butoxyethyl chloroacetate was 99.0% and the yield was 91.0 mole %.

### Example 2: Preparation of 2-Butoxyethyl Chloroacetate by Using the Recovered Unreacted Reactants, Solvent and Catalyst of Example 1

In the above reaction set up with residual mass containing para toluene sulfonic acid and unreacted mono chloroacetic acid of Example 1, 121.5 g butyl cellosolve, 94.5 g mono chloro acetic acid, recovered toluene, unreacted butyl cellosolve and small fraction of 2-butoxyethyl chloroacetate of example 1 were added to obtain a reaction mixture. The reaction mixture was heated at 86° C. and simultaneously water was removed through the Dean and Stark system to obtain a first product mixture comprising 2-butoxyethyl chloroacetate, toluene, unreacted butyl cellosolve, unreacted mono chloro acetic acid and para toluene sulfonic acid. After complete removal of water (18 ml), the first product mixture was fractionally distilled at 125° C. to recover toluene, and unreacted butyl cellosolve, to obtain a second product mixture comprising 2-butoxyethyl chloroacetate, unreacted mono chloro acetic acid and para toluene sulfonic acid. Small fraction of 2-butoxyethyl chloroacetate was also distilled out along with toluene and unreacted butyl cellosolve. Recovered toluene and butyl cellosolve was recycled in the next batch. The second product mixture was vacuum distilled at 110° C. and at 4.5 mmHg to obtain 2-butoxyethyl chloroacetate and a residual mass containing the para toluene sulfonic acid and the unreacted mono chloroacetic acid. Residual mass containing the para toluene sulfonic acid and the unreacted mono chloroacetic acid was recycled and used in the next batch.

The purity of 2-butoxyethyl chloroacetate was 99.2% and the yield was 97.0 mole %.

### Example 3: Preparation of 2-Butoxyethyl Chloroacetate by Using the Recovered Unreacted Reactants, Solvent and Catalyst of Example 2

In the above reaction set up with residual mass containing para toluene sulfonic acid and unreacted mono chloroacetic acid of Example 2, 121.5 g butyl cellosolve, 94.5 g mono chloro acetic acid, recovered toluene, unreacted butyl cellosolve and small fraction of 2-butoxyethyl chloroacetate of example 2 were added to obtain a reaction mixture. The reaction mixture was heated at 86° C. and simultaneously water was removed through the Dean and Stark system to obtain a first product mixture comprising 2-butoxyethyl chloroacetate, toluene, unreacted butyl cellosolve, unreacted mono chloro acetic acid and para toluene sulfonic acid. After complete removal of water, the first product mixture was fractionally distilled at 130° C. to recover toluene, and unreacted butyl cellosolve and to obtain a second product mixture comprising 2-butoxyethyl chloroacetate, unreacted mono chloro acetic acid and para toluene sulfonic acid. Small fraction of 2-butoxyethyl chloroacetate was also distilled out along with toluene and unreacted butyl cellosolve. Recovered toluene and butyl cellosolve was recycled in the next batch. The second product mixture was vacuum distilled at 115° C. and at 4.5 mmHg to obtain 2-butoxyethyl chloroacetate and a residual mass containing the para toluene sulfonic acid and the unreacted mono chloroacetic acid. Residual mass containing the para toluene sulfonic acid and the unreacted mono chloroacetic acid was recycled and used in the next batch.

The purity of 2-butoxyethyl chloroacetate was 99.5% and the yield was 97.0 m %.

### Example 4: Preparation of 2-Butoxyethyl Chloroacetate Using n-Hexane as a Fluid Medium in Accordance with the Present Disclosure

In a 4 neck round bottom flask with Dean and Stark apparatus, double coil condenser, overhead stirrer and reaction temperature indicator 129.8 g butyl cellosolve and 94.5 g mono chloro acetic acid were charged and reacted in the presence of 2.5 g of para toluene sulfonic acid in 100 ml n-hexane to obtain a reaction mixture. The reaction mixture was heated at 65° C. and simultaneously water was removed through the Dean and Stark system to obtain a first product mixture comprising 2-butoxyethyl chloroacetate, n-hexane, unreacted butyl cellosolve, unreacted mono chloro acetic acid and para toluene sulfonic acid. After complete removal of water (18 ml) the first product mixture was fractionally distilled at 95° C. to recover n-hexane, and unreacted butyl cellosolve and to obtain a second product mixture comprising 2-butoxyethyl chloroacetate, unreacted mono chloro acetic acid and the catalyst. Small fraction of 2-butoxyethyl chloroacetate was also distilled out along with n-hexane and unreacted butyl cellosolve. The second product mixture was vacuum distilled at 115° C. and at 4.5 mmHg to obtain 2-butoxyethyl chloroacetate and a residual mass containing the para toluene sulfonic acid and the unreacted mono chloroacetic acid. Residual mass containing the para toluene sulfonic acid and the unreacted mono chloroacetic acid was recycled and used in the next batch.

The purity of 2-butoxyethyl chloroacetate was 99.2% and the yield was 93.0 mole %.

### Example 5: Preparation of 2-Butoxyethyl Chloroacetate by Using the Recovered Unreacted Reactants, Solvent and Catalyst of Example 4

In the above reaction set up with residual mass containing para toluene sulfonic acid and unreacted mono chloroacetic acid of Example 4, 121.5 g butyl cellosolve, 94.5 g mono chloro acetic acid, recovered n-hexane, unreacted butyl cellosolve and small fraction of 2-butoxyethyl chloroacetate of example 4 were added to obtain a reaction mixture. The reaction mixture was heated at 65° C. and simultaneously water was removed through the Dean and Stark system to obtain a first product mixture comprising 2-butoxyethyl chloroacetate, n-hexane, unreacted butyl cellosolve, unreacted mono chloro acetic acid and para toluene sulfonic acid. The first product mixture was fractionally distilled at 95° C. to recover n-hexane, and unreacted butyl cellosolve and to obtain a second product mixture comprising 2-butoxyethyl chloroacetate, unreacted mono chloro acetic acid and para toluene sulfonic acid. Small fraction of 2-butoxyethyl chloroacetate was also distilled out along with n-hexane and unreacted butyl cellosolve. Recovered n-hexane and butyl cellosolve was recycled in the next batch. The second product mixture was vacuum distilled at 110° C. and at 4-5 mm Hg to obtain 2-butoxyethyl chloroacetate and a residual mass containing the para toluene sulfonic acid and the unreacted mono chloroacetic acid. Residual mass containing the para toluene sulfonic acid and the unreacted mono chloroacetic acid was recycled and used in the next batch.

The purity of 2-butoxyethyl chloroacetate was 99.0% and the yield was 97.5 mole %.

### Example 6: Preparation of 2-Butoxyethyl Chloroacetate by Using the Recovered Unreacted Reactants, Solvent and Catalyst of Example 5

In the above reaction set up with residual mass containing para toluene sulfonic acid and unreacted mono chloroacetic acid of Example 5, 121.5 g butyl cellosolve, 94.5 g mono chloro acetic acid, recovered n-hexane, unreacted butyl cellosolve and small fraction of 2-butoxyethyl chloroacetate of example 5 were added to obtain a reaction mixture. The reaction mixture was heated at 65° C. and simultaneously water was removed through the Dean and Stark system to obtain a first product mixture comprising 2-butoxyethyl chloroacetate, n-hexane, unreacted butyl cellosolve, unreacted mono chloro acetic acid and para toluene sulfonic acid. The first product mixture was fractionally distilled at 95° C. to recover n-hexane, and unreacted butyl cellosolve and to obtain a second product mixture comprising 2-butoxyethyl chloroacetate, unreacted mono chloro acetic acid and para toluene sulfonic acid. Small fraction of 2-butoxyethyl chloroacetate was also distilled out along with n-hexane and unreacted butyl cellosolve. Recovered n-hexane and butyl cellosolve was recycled in the next batch. The second product mixture was vacuum distilled at 110° C. and at 4-5 mm Hg to obtain 2-butoxyethyl chloroacetate and a residual mass containing the para toluene sulfonic acid and the unreacted mono chloroacetic acid. Residual mass containing the para toluene sulfonic acid and the unreacted mono chloroacetic acid was recycled and used in the next batch.

The purity of 2-butoxyethyl chloroacetate was 99.4% and the yield was 97.3 mole %.

### Experiment II: Preparation of 2-Butoxyethyl Chloroacetate (Aqueous Work-Up)

### Example (a): Preparation of 2-Butoxyethyl Chloroacetate Using Toluene as a Fluid Medium in Accordance with the Present Disclosure

In a 4 neck round bottom flask with Dean and Stark apparatus, double coil condenser, overhead stirrer and reaction temperature indicator, 118.0 g of butyl cellosolve and 113.4 g of mono chloro acetic acid were charged and reacted in the presence of 2.0 g of para toluene sulfonic acid (PTSA) in 100 ml toluene to obtain a reaction mixture. The reaction mixture was heated at 86° C. and simultaneously water was removed through the Dean and Stark system to obtain a product mixture comprising 2-butoxyethyl chloroacetate, toluene, unreacted mono chloro acetic acid and para toluene sulfonic acid. After complete removal of water (18 ml), the product mixture was cooled to 20° C. followed by adding 10 ml of water to obtain a first biphasic mixture comprising a first organic phase comprising 2-butoxyethyl chloroacetate along with toluene and a first aqueous phase. The first aqueous phase comprising the unreacted mono chloro acetic acid and the catalyst was separated from the first biphasic mixture and recycled in next batch. The first organic phase comprising 2-butoxyethyl chloroacetate along with toluene and traces of acid impurity was subjected to washing with 75 ml of an aqueous solution of sodium carbonate (5% w/w) to maintain a pH of 7.5 to obtain a second biphasic mixture comprising a second organic phase and a second aqueous phase. The second aqueous phase comprising the traces of mono chloro acetic acid salt and PTSA salt was separated from the second biphasic mixture to obtain the second organic phase comprising 2-butoxyethyl chloroacetate and toluene. The second organic phase was vacuum distilled at 105° C. at a pressure of 15 mmHg to recover toluene, to obtain a residual mass containing 2-butoxyethyl chloroacetate. Small fraction of 2-butoxyethyl chloroacetate was also distilled out along with toluene. The recovered toluene along with the traces of 2-Butoxy chloroacetate was recycled and used in the next batch.

The purity of 2-butoxyethyl chloroacetate was 99.0% and the yield was 96.0 mole %.

### Example (b): Preparation of 2-Butoxyethyl Chloroacetate by Using the Recovered Fluid of Example (a)

In a 4 neck round bottom flask with Dean and Stark apparatus, double coil condenser, overhead stirrer and reaction temperature indicator, 118.0 g of butyl cellosolve and 108.7 g of mono chloro acetic acid were charged and reacted in the presence of 2.0 g of para toluene sulfonic acid and recovered toluene along with the traces of 2-butoxyethyl chloroacetate of example (a) were added to obtain a reaction mixture. The reaction mixture was heated at 86° C. and simultaneously water was removed through the Dean and Stark system to obtain a product mixture comprising 2-butoxyethyl chloroacetate, toluene, unreacted mono chloro acetic acid and para toluene sulfonic acid. After complete removal of water (18 ml), the product mixture was cooled to 20° C. followed by adding 10 ml of water to obtain a first biphasic mixture comprising a first organic phase comprising 2-butoxyethyl chloroacetate along with toluene and a first aqueous phase. The first aqueous phase comprising the unreacted mono chloro acetic acid and the catalyst was separated from the first biphasic mixture and recycled in next batch. The first organic phase comprising 2-butoxyethyl chloroacetate along with toluene and traces of acid impurity was subjected to washing with 75 ml of an aqueous solution of sodium carbonate (5% w/w) to maintain a pH of 7.5 to obtain a second biphasic mixture comprising a second organic phase and a second aqueous phase. The second aqueous phase comprising the traces of mono chloro acetic acid salt and PTSA salt was separated from the second biphasic mixture to obtain the second organic phase comprising 2-butoxyethyl chloroacetate and toluene. The second organic phase was vacuum distilled at 108° C. at a pressure of 15 mmHg to recover toluene, to obtain a residual mass containing 2-butoxyethyl chloroacetate. Small fraction of 2-butoxyethyl chloroacetate was also distilled out along with toluene. The recovered toluene along with the traces of 2-Butoxy chloroacetate was recycled and used in the next batch.

The purity of 2-butoxyethyl chloroacetate was 99.05% and the yield was 97.0 mole %.

### Example (c): Preparation of 2-Butoxyethyl Chloroacetate by Using the Recovered Fluid Medium of Example (b)

In a 4 neck round bottom flask with Dean and Stark apparatus, double coil condenser, overhead stirrer and reaction temperature indicator, 118.0 g of butyl cellosolve and 108.7 g of mono chloro acetic acid were charged and reacted in the presence of 2.0 g of para toluene sulfonic acid and the recovered toluene along with the traces of 2-butoxyethyl chloroacetate of example (b) were added to obtain a reaction mixture. The reaction mixture was heated at 86° C. and simultaneously water was removed through the Dean and Stark system to obtain a product mixture comprising 2-butoxyethyl chloroacetate, toluene, unreacted mono chloro acetic acid and para toluene sulfonic acid. After complete removal of water (18 ml), the product mixture was cooled to 20° C. followed by 10 ml of water to obtain a first biphasic mixture comprising a first organic phase comprising 2-butoxyethyl chloroacetate along with toluene and a first aqueous phase. The first aqueous phase comprising the unreacted mono chloro acetic acid and the catalyst was separated from the first biphasic mixture and recycled in next batch. The first organic phase comprising 2-butoxyethyl chloroacetate along with toluene and traces of acid impurity was subjected to washing with 75 ml of an aqueous solution of sodium carbonate (5% w/w) to maintain a pH of 7.5 to obtain a second biphasic mixture comprising a second organic phase and a second aqueous phase. The second aqueous phase comprising the traces of mono chloro acetic acid salt and PTSA salt was separated from the second biphasic mixture to obtain the second organic phase comprising 2-butoxyethyl chloroacetate and toluene. The organic phase was vacuum distilled at 108° C. at a pressure of 15 mmHg to recover toluene and to obtain a residual mass containing 2-butoxyethyl chloroacetate. Small fraction of 2-butoxyethyl chloroacetate was also distilled out along with toluene. The recovered toluene along with the traces of 2-Butoxy chloroacetate was recycled and used in the next batch.

The purity of 2-butoxyethyl chloroacetate was 99.5% and the yield was 97.5 mole %.

### Example (d): Preparation of 2-Butoxyethyl Chloroacetate Using n-Hexane as a Fluid Medium in Accordance with the Present Disclosure

The same procedure of Example (a) was followed except that 100 ml of n-hexane was used instead of toluene to obtain 2-butoxyethyl chloroacetate.

The purity of 2-butoxyethyl chloroacetate was 99.5% and the yield was 97.0 mole %.

### Example (e): Preparation of 2-Butoxyethyl Chloroacetate by Using the Recovered Fluid Medium and Recovered Catalyst of Example (e)

The same procedure of Example (a) was followed except that the recovered n-hexane along with the traces of 2-butoxyethyl chloroacetate of example (d) was used instead of toluene to obtain 2-butoxyethyl chloroacetate.

The purity of 2-butoxyethyl chloroacetate was 99.4% and the yield was 97.2 mole %.

### Example 6: Preparation of 2-Butoxyethyl Chloroacetate by Using the Recovered Fluid Medium and Recovered Catalyst of Example (e)

The same procedure of Example (a) was followed except that the recovered n-hexane along with the traces of 2-butoxyethyl chloroacetate of example (e) was used instead of toluene to obtain 2-butoxyethyl chloroacetate.

The purity of 2-butoxyethyl chloroacetate was 99.6% and the yield was 97.0 mole %.

### Experiment III: Preparation of Triclopyr-Butotyl

### Example (i): Preparation of Triclopyr-Butotyl in Accordance with the Present Disclosure

In 1 litre capacity reaction vessel equipped with temperature sensor, condenser and overhead stirrer, 194.5 g of 2-butoxyethyl chloroacetate and 238.5 g of sodium 3,5,6-trichloropyridin-2-olate were charged and reacted by using 5 g of sodium bicarbonate in the presence of 8 g of tetrabutylammonium bromide (catalyst) at 65° C. for 10 hours to obtain a reaction mixture. Reaction progress was monitored by Gas Chromatography and when 2-Butoxyethyl chloroacetate content became <1.0% w/w, the reaction mixture was cooled to 60° C. followed by adding 400 ml n-hexane to obtain a reaction mass. The reaction mass was further cooled to 30° C. and filtered to obtain a residue comprising insoluble impurities and sodium chloride salt and a filtrate comprising triclopyr-butotyl. The residue was washed with 50 ml n-hexane and the washings were mixed with the filtrate. The filtrate was cooled to 15° C. to obtain a cooled filtrate. The cooled filtrate was washed with 50 ml of 70% w/w sulfuric acid for 30 minutes to obtain a biphasic mixture containing a top organic layer comprising triclopyr-butotyl and a bottom aqueous layer comprising sulfuric acid solution. The aqueous layer comprising the acid solution is separated to obtain the organic layer comprising the triclopyr-butotyl and n-hexane. The organic layer was washed with 10 g of NaHCO₃ dissolved in 200 ml of 3% NaOCl solution followed by further washing with 2% w/w NaHSO₃ solution to obtain a washed organic layer comprising the triclopyr-butotyl and n-hexane. The washed organic layer was distilled at 100° C. to obtain a residual mass containing the triclopyr-butotyl and to recover n-hexane. The residual mass was vacuum dried at 100° C. at a pressure of 6 mmHg to obtain the triclopyr-butotyl.

The purity of triclopyr-butotyl was 99.0% and the yield was 87.0 mole %.

The spent H₂SO₄ generated in the above process was diluted to 50% w/w and extracted with toluene to remove organic impurities in it. The diluted and cleaned H₂SO₄ was concentrated to get 80% w/w and recycled in the next batch.

### Examples (ii) to (vi): Preparation of Triclopyr-Butotyl by Varying the Process Parameters

The same procedure of Example (i) was followed except the varying process parameters as given in Table 1 below.

TABLE 1

| Process parameters | Eample (i) | | Eample (ii) | | Eample (iii) | | Eample (iv) | | Eample (v) | | Example (vi) | |
|---|---|---|---|---|---|---|---|---|---|---|---|---|
| 2-Butoxyethyl | 194.5 | g | 194.5 | g | 194.5 | g | 194.5 | g | 194.5 | g | 194.5 | g |
| chloroacetate | | | | | | | | | | | | |
| sodium 3,5,6- | 238.5 | g | 238.5 | g | 238.5 | g | 238.5 | g | 238.5 | g | 238.5 | g |
| trichloropyridin- | | | | | | | | | | | | |
| 2-olate | | | | | | | | | | | | |
| NaHCO₃ | 5 | g | 5 | g | 5 | g | 5 | g | 5 | g | 5 | g |
| Catalyst | Tetrabutylammonium | | TBAB | | TBAB | | TBAB | | TBAB | | Benzyl triethyl | |
| | bromide (TBAB) | | 8 g | | 4 g | | 6 g | | 8 g | | ammonium chloride | |
| | 8 g | | | | | | | | | | 15 g | |
| Heating | 65° | C. | 75° | C. | 75° | C. | 77° | C. | 50° | C. | 75° | C. |
| temperature | | | | | | | | | | | | |
| (First | | | | | | | | | | | | |
| predetermined | | | | | | | | | | | | |
| temperature) | | | | | | | | | | | | |
| Reaction time | 10 | hours | 4 | hours | 10 | hours | 6 | hours | 16 | hours | 12 | hours |
| period | | | | | | | | | | | | |
| Cooling | 60° | C. | 60° | C. | 60° | C. | 60° | C. | 50° | C. | 60° | C. |
| Temperature | | | | | | | | | | | | |
| (Second | | | | | | | | | | | | |
| predetermined | | | | | | | | | | | | |
| temperature) | | | | | | | | | | | | |
| Yield | 87.0 | mole %. | 87.0 | mole %. | 86.0 | mole %. | 87.0 | mole %. | 87.0 | mole %. | 70.0 | mole %. |

| | | | | | | |
|---|---|---|---|---|---|---|
| Purity | 99.0% | 98.5% | 98.5% | 98.5% | 98.5% | 98.0% |

From the examples (i) to (vi), it was observed that when the reaction was carried out at high temperature (75° C. to 77° C.), the reaction rate was faster and hence, the reaction time required was less (4 hours to 6 hours). Further, when the catalyst loading was more, the reaction rate became faster even at low temperature (50° C. to 65° C.). However, there was no difference observed in the yield and purity of the triclopyr-butotyl. Furthermore, in the example (vi), wherein benzyl triethyl ammonium chloride was used as a catalyst, the amount of the catalyst required was more as compared to the examples (i) to (v), wherein TBAB was used as the catalyst. Further, the yield (70.0 mole %) and purity (98.0%) of the triclopyr-butotyl in example (vi) was lower as compared to the yield and purity of the triclopyr-butotyl in the examples (i) to (v).

### Technical Advancements

The present disclosure described hereinabove has several technical advantages including, but not limited to, the realization of a process for the preparation of 2-butoxyethyl chloroacetate, that:
is environment friendly and economic as the fluid media and the acid are recycled;
is simple and efficient; and
provides triclopyr-butotyl in high yield and high purity.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

The numerical values given for various physical parameters, dimensions and quantities are only approximate values and it is envisaged that the values higher than the numerical value assigned to the physical parameters, dimensions and quantities fall within the scope of the invention unless there is a statement in the specification to the contrary.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values given for various physical parameters, dimensions, and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

1. A process for preparing triclopyr-butotyl, said process comprising the following steps:
i. reacting 2-butoxyethyl chloroacetate with a compound selected from sodium 3,5,6-trichloropyridin-2-olate, potassium 3,5,6-trichloropyridin-2-olate and ammonium 3,5,6-trichloropyridin-2-olate in a predetermined molar ratio by using a first base in the presence of a first catalyst under stirring at a first predetermined temperature for a first predetermined time period to obtain a reaction mixture;
ii. adding a predetermined amount of a first fluid medium to said reaction mixture at a second predetermined temperature followed by cooling to a third predetermined temperature to obtain a reaction mass;
iii. filtering said reaction mass to obtain a residue comprising insoluble impurities and a salt; and a filtrate comprising triclopyr-butotyl;
iv. cooling said filtrate to a fourth predetermined temperature to obtain a cooled filtrate;
v. washing said cooled filtrate with a predetermined concentration of sulfuric acid for a second predetermined time period to obtain a biphasic mixture containing a top organic layer comprising triclopyr-butotyl and a bottom aqueous layer comprising sulfuric acid solution;
vi. separating said aqueous layer comprising said acid solution to obtain said organic layer comprising said triclopyr-butotyl and said first fluid medium;
vii. washing said organic layer with at least one salt solution followed by a second base solution to obtain a washed organic layer comprising said triclopyr-butotyl and said first fluid medium;
viii. distilling said washed organic layer at a fifth predetermined temperature to recover said first fluid medium, to obtain a residual mass containing said triclopyr-butotyl; and
ix. vacuum drying said residual mass at a sixth predetermined temperature and at a predetermined pressure to obtain said triclopyr-butotyl.

2. The process as claimed in claim 1, wherein said recovered first fluid medium and said aqueous layer comprising said acid solution are recycled to a next batch.

3. The process as claimed in claim 1, wherein said predetermined molar ratio of 2-butoxyethyl chloroacetate to sodium 3,5,6-trichloropyridin-2-olate is in the range of 1:1 to 1:1.5.

4. The process as claimed in claim 1, wherein said first catalyst is selected from the group consisting of tetrabutylammonium bromide (TBAB) and benzyl triethyl ammonium chloride.

5. The process as claimed in claim 1, wherein said first base and said second base are independently selected from sodium carbonate, sodium bicarbonate, potassium carbonate, potassium bicarbonate, sodium hydroxide and potassium hydroxide.

6. The process as claimed in claim 1, wherein said first fluid medium is selected from the group consisting of toluene, n-hexane and benzene; and wherein said predetermined amount of said fluid medium is in the range of 300 ml/mole of 2-butoxyethyl chloroacetate to 1000 ml/mole of 2-butoxyethyl chloroacetate.

7. The process as claimed in claim 1, wherein said first predetermined temperature is in the range of 45° C. to 90° C. and said first predetermined time period is in the range of 3 hours to 20 hours; said second predetermined temperature is in the range of 45° C. to 60° C.; said third predetermined temperature is in the range of 25° C. to 35° C.; said fourth predetermined temperature is in the range of 0° C. to 20° C.; said fifth predetermined temperature is in the range of 60° C. to 120° C.; said sixth predetermined temperature is in the range of 90° C. to 120° C. and said predetermined pressure is in the range of 4 mmHg to 8 mmHg.

8. (canceled)

9. The process as claimed in claim 1, wherein said predetermined concentration of sulfuric acid is in the range of 60% w/w to 80% w/w; and said second predetermined time period is in the range of 20 minutes to 60 minutes; and said salt solution is prepared by using an alkali metal salt selected from sodium hypochlorite (NaOCl), sodium bisulfite (NaHSO₃) and sodium sulfite (Na₂SO₃).

10. (canceled)

11. (canceled)

12. The process as claimed in claim 1, wherein a yield of triclopyr-butotyl is in the range of 85 mole % to 90 mole % and a purity is in the range of 98% to 99%.

13. The process as claimed in claim 1, wherein 2-butoxyethyl chloroacetate is prepared by the following sub-steps:
i. reacting butyl cellosolve with mono chloro acetic acid in a predetermined molar ratio in the presence of a second catalyst in a second fluid medium to obtain a reaction mixture;
ii. heating said reaction mixture at a seventh predetermined temperature and simultaneously distilling out water azeotropically to obtain a first product mixture comprising 2-butoxyethyl chloroacetate, said second fluid medium, unreacted butyl cellosolve, unreacted mono chloro acetic acid and said second catalyst; and
iii. isolating and purifying 2-butoxyethyl chloroacetate from said first product mixture.

14. The process as claimed in claim 13, wherein said predetermined molar ratio of said butyl cellosolve to said mono chloroacetic acid is in the range of 1:0.5 to 1:1.5.

15. The process as claimed in claim 13, wherein said second catalyst is selected from the group consisting of para toluene sulfonic acid (PTSA) and methane sulphonic acid; and wherein said second catalyst is present in an amount in the range of 1 mole % to 2 mole % with respect to the amount of butyl cellosolve.

16. The process as claimed in claim 13, wherein said second fluid medium is selected from the group consisting of toluene, n-hexane, cyclohexane, benzene and chlorobenzene.

17. The process as claimed in claim 13, wherein said seventh pre-determined temperature is in the range of 50° C. to 130° C.

18. The process as claimed in claim 13, wherein said isolation and said purification of 2-butoxyethyl chloroacetate is carried out by;
either;
a) fractionally distilling said first product mixture at an eighth pre-determined temperature to recover said second fluid medium and said unreacted butyl cellosolve, to obtain a second product mixture comprising 2-butoxyethyl chloroacetate, said unreacted mono chloro acetic acid and said second catalyst; and
b) vacuum distilling said second product mixture at a ninth predetermined temperature and at a first predetermined pressure to obtain 2-butoxyethyl chloroacetate and a residual mass containing said second catalyst and said unreacted mono chloroacetic acid;
or;
a) adding water to said first product mixture to obtain a first biphasic mixture containing a first organic phase and a first aqueous phase, wherein said first aqueous phase comprises said unreacted mono chloro acetic acid and said second catalyst and said first organic phase comprises 2-butoxyethyl chloroacetate and said second fluid medium;
b) separating said first aqueous phase from said first biphasic mixture to obtain said first organic phase comprising 2-butoxyethyl chloroacetate and said second fluid medium;
c) washing said first organic phase with an aqueous third base by maintaining a pH in the range of 6 to 9 to obtain a second biphasic mixture containing a second organic phase and a second aqueous phase followed by separating said second aqueous phase from said second biphasic mixture to obtain said second organic phase containing 2-butoxyethyl chloroacetate and said second fluid medium; and
d) vacuum distilling said second organic phase at a tenth predetermined temperature and at a second predetermined pressure to recover said second fluid medium, to obtain a residual mass containing 2-butoxyethyl chloroacetate.

19. The process as claimed in claim 18, wherein said recovered second fluid medium along with said unreacted butyl cellosolve and said residual mass containing said catalyst and said unreacted mono chloroacetic acid are recycled to a next batch.

20. The process as claimed in claim 18, wherein said eighth pre-determined temperature is in the range of 90° C. to 130° C.; and said ninth pre-determined temperature is in the range of 100° C. to 120° C.; and said tenth pre-determined temperature is in the range of 100° C. to 120° C.; said first predetermined pressure is in the range of 4 mmHg to 5 mmHg; and said second predetermined pressure is in the range of 10 mmHg to 20 mmHg.

21. (canceled)

22. (canceled)

23. The process as claimed in claim 18, wherein said third base is selected from the group consisting of sodium carbonate, sodium bicarbonate, potassium carbonate, potassium bicarbonate, sodium hydroxide and potassium hydroxide.

24. The process as claimed in claim 18, wherein a small fraction of 2-butoxyethyl chloroacetate is distilled out along with said recovered second fluid medium; and wherein said recovered second fluid medium along with said small fraction of 2-butoxyethyl chloroacetate are recycled to a next batch.

25. (canceled)

26. (canceled)

27. The process as claimed in claim 18, wherein a yield of 2-butoxyethyl chloroacetate is in the range of 90 mole % to 99 mole % and a purity is in the range of 98% to 99.9%.
